(19) United States
(12) Patent Application Publication
(10) Pub. No.: US 2024/0083889 A1
(43) Pub. Date: Mar. 14, 2024

(54) Scalable process for the preparation of a GlyT-1 Inhibitor

(71) Applicant: Boehringer Ingelheim International GmbH, Ingelheim am Rhein (DE)

(72) Inventors: Katrin BAER, Mainz (DE); Gisela BODENBACH, Niederburg (DE); Jack Delbert BROWN, Moseley, VA (US); Rosemarie Karoline COLLET, Bad Kreuznach (DE); Daniel HERKOMMER, Mainz (DE); Rogelio P. FRUTOS, Sandy Hook, CT (US); Joe Ju GAO, Southbury, CT (US); Julia Regina GRIMM, Gau-Algesheim (DE); Sandra KOCH, Wiesbaden (DE); Sonia RODRIGUEZ, Avon, CT (US); Svetlana SEHL-OLLENBERGER, Mainz (DE); Joshua Daniel SIEBER, Richmond, VA (US); Thomas G. TAMPONE, Southbury, CT (US); Ulrich THEIS, Nastaetten (DE); Dirk WEBER, Mainz (DE); Erik WEIS, Ingelheim am Rhein (DE); Anke WILD, Mainz (DE)

(21) Appl. No.: 18/237,423

(22) Filed: Aug. 24, 2023

(30) Foreign Application Priority Data
Aug. 24, 2022 (EP) 22 192 010.1

(51) Int. Cl. C07D 413/04

(52) U.S. Cl. CPC C07D 413/04

(57) ABSTRACT

This invention relates to a synthetic method for the preparation of Compound 1 and precursors thereof. Compound 1 is prepared via reaction of isoxazole 2 with phenylether (R)-3-ONa.

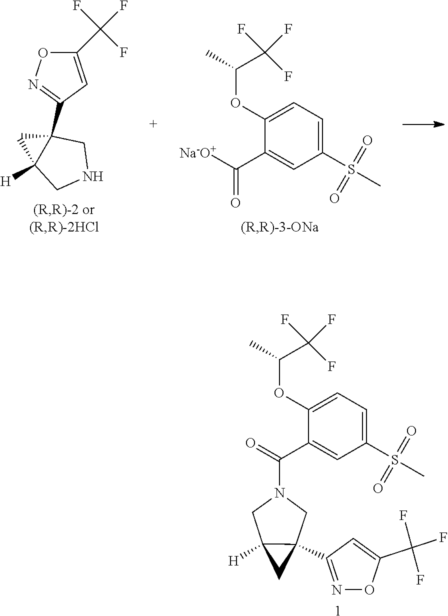

### FIELD OF THE INVENTION

This invention relates to a scalable synthesis process for the preparation of a specific bicyclo[3.1.0]hex-3-yl methanone (Compound 1) having inhibitory actions on glycine transporter 1 (GlyT-1). The process is suitable for the manufacture of Compound 1 on multikilogram scale.

### BACKGROUND

Dysfunction of glutamatergic neurotransmission mediated by N-methyl-D-aspartate (NMDA) receptors is implicated in the aetiology of CIAS. Various strategies for enhancing glutamatergic transmission have been investigated in patients with schizophrenia; one approach involves increasing synaptic levels of glycine, a coagonist required for NMDA receptor-mediated signaling. Inhibitors of glycine transporter-1 (GlyT1) are thought to elevate synaptic glycine levels and consequently enhance glutamatergic neurotransmission and downstream neuroplasticity processes.

Therefore, activation of NMDA receptors via GlyT1 inhibition may offer options to treat psychosis, schizophrenia (positive, negative and cognitive symptoms), dementia and other diseases in which cognitive processes are impaired, such as attention deficit disorders, Alzheimer's disease, or other neurological and psychiatric disorders. Inhibition of GlyT1 is of high interest, in particular with respect to cognitive impairment associated with Schizophrenia.

Compound 1 is described in WO2013/017657. Compound 1 is a potent and selective GlyT1 inhibitor and has been investigated in a phase II study in patients with schizophrenia.

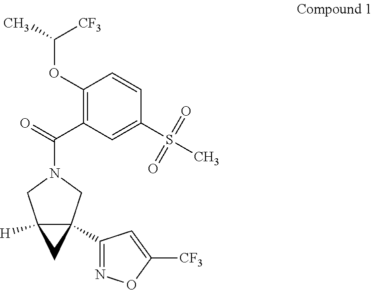 [SMILES: C[C@@H](Oc1ccc(S(C)(=O)=O)cc1C(=O)N1C[C@@H]2C[C@]2(c2cc(C(F)(F)F)on2)C1)C(F)(F)F]

([5-(methylsulfonyl)-2-{[(2R)-1,1,1-trifluoropropan-2-yl]oxy}phenyl]{(1R,5R)-1-[5-(trifluoromethyl)-1,2-oxazol-3-yl]-3-azabicyclo[3.1.0]hex-3-yl}methanone)

WO2013/017657 describes the preparation of Compound 1 and has been prepared according to Scheme 1.

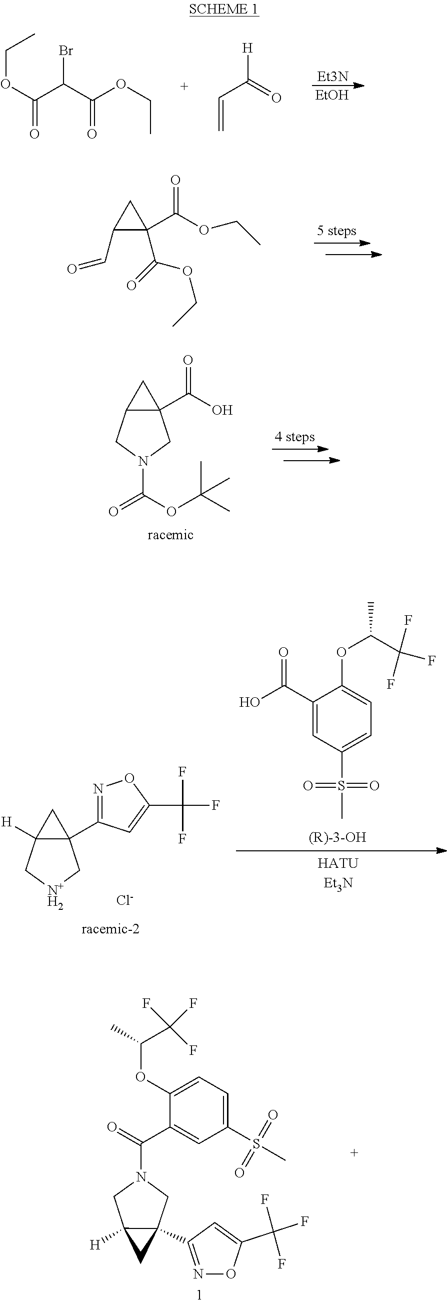

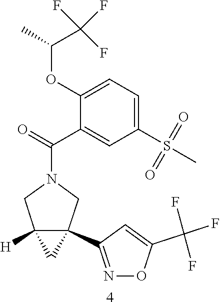

In the initial route racemic isoxazole 2 and chiral (R)-3 were prepared as the two main building blocks and then coupled to give an equal mixture of diastereomers 1 and 4 that could only be resolved through a labor intensive preparative HPLC chromatography.

Example 1 of WO2008/107334 describes the synthesis of (S)-3-OH (sub-kilogram scale) via coupling of 2-fluoro-5-methanesulfonyl-benzoic acid (10) with (S)-trifluoro-isopropanol in an autoclave and with cesium carbonate as base. Alternatively, a procedure in a double-jacket vessel is described using 10 and (S)-trifluoro-isopropanol in the presence of KOtBu.

WO2008/107334 further describes the conversion of 2-fluorobenzoic acid to 10 via a 2-step procedure via the corresponding sulfonylchloride in a total yield of 45%.

### SUMMARY OF THE INVENTION

An object of the present invention is a safe and efficient scalable manufacturing process that provides access to Compound 1 on a multikilogram scale. The object has been achieved by the provision of the processes as described herein.

### BRIEF DESCRIPTION OF THE SEVERAL VIEW OF THE FIGURES

FIG. 1 shows an overview of the entire process for making Compound 1 as described herein.

### DESCRIPTION OF THE PROCESS

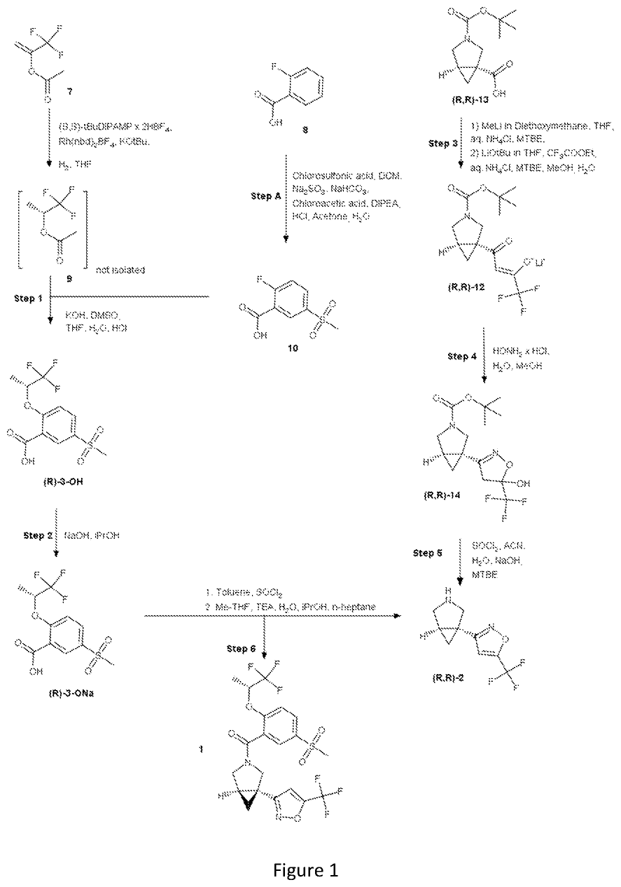

The processes according to the present invention is summarized in FIG. 1. Description of the individual steps are described below.

A Rh-catalyzed asymmetric hydrogenation of 7 using the solid and air-stable ligand t-Bu-SMS-Phos.2HBF₄ (8) provides (R)-1,1,1-trifluoropropan-2-ylacetate (9) in excellent yield and enantioselectivity (99% yield and 98-99% ee) (Scheme 2). [J. Sieber, S. Rodriguez, R. Frutos, F. Buono, Y. Zhang, N. Li, B. Qu, A. Premasiri, Z. Li, Z. Han, Y. Xu, D. Byrne, N. Haddad, J. Lorenz, N. Grinberg, D. Kurouski, H. Lee, B. Narayanan, L. Nummy, J. Mulder, J. Brown, A. Granger, M. Krawiec, Z. Williams, S. Pennino, J. Song, A. Hossain, N. Yee, C. Busacca, F. Roschangar, Y. Xin, Z. Mao, X. Zhang, Y. Hong, C. H. Senanayake, J. Org. Chem. 2018, 83, 1448.] The product can be distilled directly from the hydrogenation reaction mixture providing a distillate containing exclusively a solution of 9 in THF that can be used in the next process stage. Surprisingly, residual Rh catalyst or phosphor ligand do not negatively affect the subsequent nucleophilic aromatic substitution and thus it is possible to use the reaction mixture of the hydration directly—without distillation—for the synthesis of phenylether (R)-3-OH). Purging of the remaining Rh catalyst and phosphor ligand is possible in the downstream steps without impacting the quality of the final API.

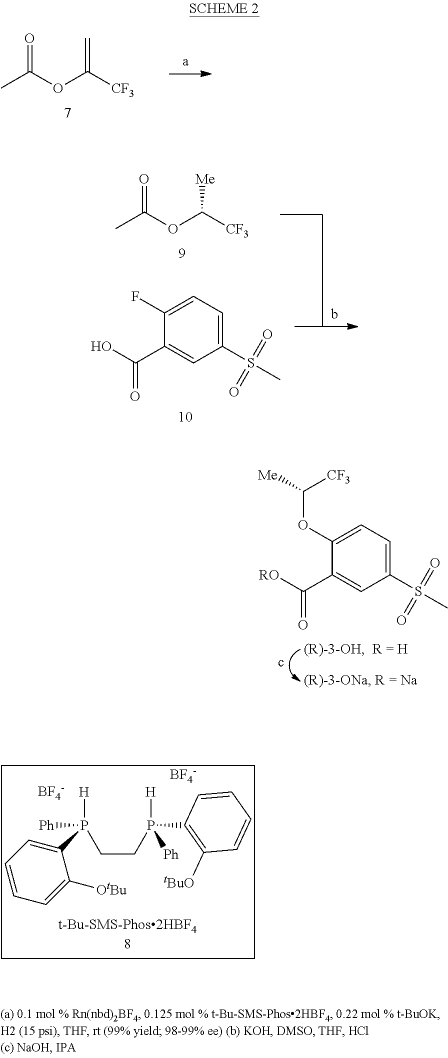

### Step A: Synthesis of methylsulfone 10

2-Fluorobenzoic acid is treated with chlorosulfonic acid in CH₂Cl₂, followed by aqueous work-up. The crude organic layer is treated with sodium sulfite and sodium hydrogen carbonate followed by addition of diisopropylamine and chloroacetic acid.

Crude methylsufone 10 is recrystallized from acetonitrile.

### Step 1: Synthesis of phenylether (R)-3-OH

Acetate 9 is de-acetylated in-situ with a strong base such as n-BuLi or KOH to provide the alkoxide of trifluoropropanol that is added to 10 in a SNAr manner to give 3 in good yield.

The use of acetate 9 avoids the problems associated with isolation of volatile trifluoro-isopropanol (6).

The crude solution of acetate 9 obtained from the hydrogenation of 7 is used without prior distillation of acetate 9.

### Step 2: Synthesis of sodium salt 3-ONa

Carboxylic acid (R)-3 is subsequently converted to its crystalline sodium salt (R)-3-ONa to reject potential impurities and ensure the quality of the material produced was consistently within the required specifications, specifically enantiomeric excess of (R)-3-ONa can further be increased and consequently formation of diastereoisomers of Compound 1 be reduced.

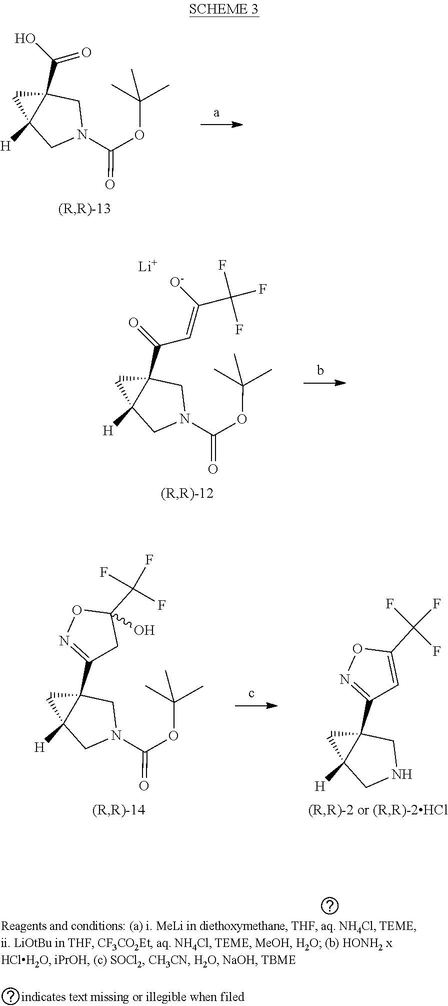 [SMILES: CC(C)(C)OC(=O)N1C[C@@H]2C[C@]2(C(=O)O)C1 ; CC(C)(C)OC(=O)N1C[C@@H]2C[C@]2(C(=O)/C=C(\[O-])C(F)(F)F)C1 ; [Li+] ; CC(C)(C)OC(=O)N1C[C@@H]2C[C@]2(C2=NOC(O)(C(F)(F)F)C2)C1 ; FC(F)(F)c1cc([C@@]23CNC[C@@H]2C3)no1]

### Step 3: Synthesis of enolate (R,R)-12

Carboxylic acid (R,R)-13 can be prepared via the reaction of (R)-epichlorohydrin and diethyl malonate through a modification of the procedure described by Lee and coworkers (J. Org. Chem. 2007, 72,7390) followed by reduction in-situ with NaBH₄/ethanol to give the corresponding diol (WO2010/007032, Shuto et al. Org. Lett, 2013, 15, 1686], which can be converted to the corresponding bis-mesylate (WO2010/0070325, WO2016/0144637). Further conversion with aqueous ammonia and protection with Boc anhydride provides the ethyl ester of (R,R)-13, which can be hydrolysed with LiOH.

Carboxylic acid (R,R)-13 is treated with MeLi to give the corresponding methyl ketone after protonation. The synthesis can be directly continued by deprotonation with lithium tert-butoxide and condensation with ethyl trifluoroacetate to provide enolate (R,R)-12, which is isolated as a solid.

### Step 4: Synthesis of dihydroisoxazole (R,R)-14

Enolate (R,R)-12 is reacted with hydroxylamine hydrochloride to provide dihydroisoxazole (R,R)-14 as an epimeric mixture.

### Step 5: Synthesis of isoxazole (R,R)-2

Dihydroisoxazole (R,R)-14 is dehydrated with thionyl chloride to give directly deprotected is isoxazole (R,R)-2, which can be isolated as hydrochloride salt ((R,R)-2·HCl) or as free base ((R,R)-2).

Use of the free base (R,R)-2 has the advantage that its synthesis is simplified (no additional addition of HCl/solutions containing HCl) and more cost-efficient without negative impact with regard to impurities or isolation.

### Step 6: Coupling to Compound 1

WO 2013/017657 describes the use of HATU/Et₃N in DMF to accomplish the final coupling of the two fragments (R,R)-2 and (R)-3-OH. However, other reagents such as isobutyl chloroformate (IBCF), propanephosphonic acid anhydride (T3P) or SOCl₂ were found to mediate the coupling as efficiently and at a lower cost.

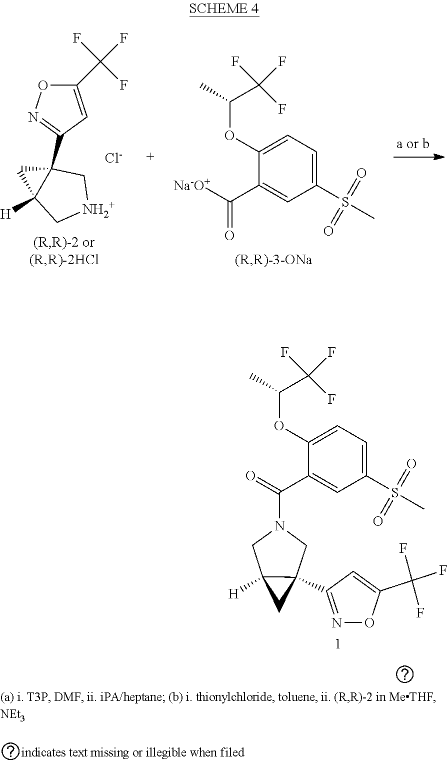

### Procedure A

The coupling of (R,R)-2·HCl and (R)-3-OH is carried out on a pilot plant scale using T3P (50% solution in EtOAc) in DMF. The resulting product can be isolated after crystallization from isopropanol and heptane.

### Procedure B and C

Thionyl chloride is added to a suspension of the sodium salt of phenylether (R)-3-ONa in toluene. The suspension is heated to 80° C. and further stirred and then concentrated under vacuum to yield the acid chloride (R)-3-Cl.

Isoxazole (R,R)-2 is dissolved in Me-THF in a separate reaction vessel, and NEt₃ is added. The resulting solution is dosed to the acid chloride solution. After complete reaction the mixture is poured into water, and the two phases are separated. The organic phase is concentrated. Isopropanol is added and again concentrated.

The remaining solution is heated to about 70° C. Heptane (or alternatively isopropanol/heptane) and seeding crystals are added and slowly cooled to obtain Compound 1 in the form of crystals.

### EMBODIMENTS OF THE INVENTION

### Step A: Synthesis of methylsulfone 10

According to a first aspect of the invention, a process to manufacture methylsulfone 10 is provided, wherein the process comprises reacting 2-fluorobenzoic acid with chlorosulfonic acid followed by treatment with sodium sulfite and chloroacetic acid. The process preferably is performed without isolation of any intermediates, e.g. preferably without isolation of the corresponding sulfonylchloride.

Crude methylsulfone 10 can be re-crystallized from acetonitrile.

### Step 1: Synthesis of phenylether 3-OH

According to a further aspect of the invention, a process to manufacture either (R)- or (S)-5-(methylsulfonyl)-2-((1,1,1-trifluoropropan-2-yl)oxy)benzoic acid is provided, wherein the process comprises reacting either (R)- or (S)-1,1,1-trifluoropropan-2-ylacetate (9) in the presence of methylsulfone 10 and a strong base.

In an embodiment the strong base is selected from the group consisting of n-BuLi, KOH, NaOH, KOtBu.

In a specific embodiment the strong base is KOH.

In an embodiment DMSO, THF, NMP or mixtures thereof is used as solvent. Preferably, DMSO or a mixture of DMSO and THF is used as solvent.

In an embodiment, a solution of methylsulfone 10 in DMSO is added to a solution of either (R)- or (S)-1,1,1-trifluoro-2-propanol (6), wherein the solution of either (R)- or (S)-1,1,1-trifluoro-2-propanol (6) is prepared in situ by addition of acetate (R)- or (S)-9 to a solution of the strong base (preferably KOH) in DMSO and THF.

With n-BuLi the formation of 5-methyl-5-nonanol is observed as a side product, which may hamper the isolation process.

The process as described above using KOH as base allows removal of KF and/or KOAc by simple filtration after the conversion of methylsulfone 10 is complete and the mixture cooled to 20° C. Therefore, the formation of HF during work-up (acidification) is largely avoided.

According to a preferred embodiment, the crude solution of acetate 9 obtained from the hydrogenation of 7 is used, i.e. the reaction mixture obtained from the Rh-catalyzed asymmetric hydrogenation of 7 is subjected to the coupling with methylsulfone 10 without distillation of acetate 9, or other treatment to reduce the Rh content.

### Step 2: Synthesis of sodium salt (R)-3-ONa

According to a further aspect, phenylether (R)-3 is converted into the sodium salt (R)-3-ONa and isolated as a crystalline compound.

In an embodiment, NaOH is used in the conversion. In a specific embodiment, the conversion is performed with NaOH in iPrOH.

Due to the conversion to the sodium salt (R)-3-ONa, the process allows facile isolation of the crystalline compound and provides efficient rejection of potential impurities and enrichment of the desired enantiomer.

### Step 3: Synthesis of enolate 12

According to a further aspect, carboxylic acid 13 is reacted with MeLi followed by quenching, deprotonation and addition of ethyl trifluoroacetate to obtain enolate 12.

In an embodiment, MeLi in diethoxymethane is used.

In an embodiment, aq. NH₄Cl is used for quenching.

In an embodiment, LiOtBu or LiN(TMS)₂ is used for the deprotonation, preferably LiOtBu.

### Step 4: Synthesis of dihydroisoxazole 14

According to a further aspect, enolate 12 is converted to dihydroisoxazole 14 with hydroxylamine.

In an embodiment, the hydroxylamine is applied in the form of its hydrochloric salt.

In an embodiment, the conversion to 14 is performed in iPrOH, MeOH or in a mixture of iPrOH and water.

### Step 5: Synthesis of isoxazole 2

According to a further aspect, dihydroisoxazole 14 is converted to isoxazole (R,R)-2 as free base.

In an embodiment, the dehydrating agent is selected from the group consisting of SOCl₂, and (COCl)₂.

In a specific embodiment, the dehydrating agent is SOCl₂.

In an embodiment, the solvent is selected from CH₃CN, H₂O, MTBE or mixtures thereof.

### Step 6: Coupling to Compound 1

### Procedure A

According to a further aspect, a solution of T3P in EtOAc is added to a solution of phenylether (R)-3-ONa-OH in DMF followed by addition of a solution of isoxazole 2 in DMF.

### Procedure B and C

According to one aspect, the acid chloride (R)-3-Cl is used in the reaction with isoxazole 2 to provide Compound 1.

According to a further aspect, the sodium salt (R)-3-ONa is converted to the corresponding acid chloride ((R)-3-Cl), e.g. by reacting with SOCl₂ or (COCl)₂, and then coupled with isoxazole 2 to provide Compound 1.

In an embodiment, SOCl₂ is used for the conversion to the acid chloride. Toluene, DMF, THF or CH₂Cl₂ may be used as solvent. Toluene is preferably used as solvent for the conversion to the acid chloride.

In an embodiment, a solution of isoxazole 2 is added to a solution of the acid chloride in toluene.

In a more specific embodiment, a solution of isoxazole 2 in Me-THF (and optionally in the presence of triethylamine) is added to a solution of the acid chloride.

### List of Abbreviations

δ Chemical Shift
br broad
¹³C Carbon 13
d Doublet
DEM Diethoxymethane
DMF N,N-Dimethylformamide
DMSO Dimethyl sulfoxide
DMSO-d₆ Deuterium Dimethyl Sulfoxide
ESI Electrospray ionization
¹H Proton
HRMS High resolution mass spectrometry
LC/MSD Liquid chromatography/mass selective detector
m Multiple
m/z Mass-to-Charge Ratio
Me-THF 2-Me-THF
MHz Mega Hertz
MS Mass Spectroscopy
MTBE Methyl tert-butyl ether
NMP N-Methylpyrrolidinone
NMR Nuclear Magnetic Resonance Spectroscopy
q Quartet
RT Room Temperature
s Singlet
t Triplet
T3P Propanephosphonic acid anhydride
THF Tetrahydrofuran
TMS Tetramethylsilane

### Synthetic Examples and Experimental Data

Unless otherwise specified, all reactions were carried out under an atmosphere of nitrogen. NMR spectra were recorded on a 400 MHz NMR spectrometer. Chemical Shifts are reported in ppm relative to tetramethylsilane; coupling constants (J) are reported in hertz, referred to as apparent peak multiplicities, and may not necessarily reflect true coupling constants. High resolution mass spectral data was acquired using a LC/MSD TOF (time-of-flight) mass spectrometer in an electrospray positive ionization mode via flow injection. The commercially available starting materials were used as received without further purification.

### Step A: Preparation of methylsulfone 10

Vessel 1 is charged with 2-fluorobenzoic acid (126 kg) and dichloromethane, stab. with amylene (335 kg). The resulting suspension is heated to reflux. Chlorosulfonic acid is dosed to the reaction mixture. First a small amount of chlorosulfonic acid (90 kg) is charged, then the main amount (277 kg) is charged under reflux conditions. In parallel dichloromethane is distilled off to reach a reaction temperature of higher than 90° C. (between 90 and 100° C.) at the end of the chlorosufonic acid addition. The vessel content is stirred for at least 60 min at an internal temperature of preferable 95° C. The reaction mixture is then cooled to an internal temperature of 10-20° C.

In vessel 2 a mixture of water (1008 kg) and dichloromethane (922 kg) is cooled to 0-10° C. The content of vessel 1 is dosed to vessel 2 at an internal temperature below 25° C. and vessel 1 is flushed with dichloromethane (512 kg) into vessel 2. The content of vessel 2 is stirred for around 15min at 15-25° C. The layers are then allowed to separate, and the aqueous layer is extracted again with dichloromethane (461 kg). The organic layers are combined in vessel 3.

Water (504 kg), sodium sulfite (101 kg) and sodium hydrogen carbonate (151 kg) are charged to vessel 4 and heated to an inner temperature of preferable 55° C. The content of vessel 3 is dosed over at least 60 min to vessel 4 maintaining the inner temperature of vessel 4 preferably around 55° C. Vessel 3 is rinsed with dichloromethane (169 kg) into vessel 4 (during dosing of the dichloromethane solution as well as during stirring at 50-60° C. dichloromethane is distilled off in parallel).—The reactor content of vessel 4 is stirred for at least 60 min at 50-60° C. Reactor content of vessel 4 is heated to 65° C. and diisopropylamine 130 kg) is charged to vessel 4 at an internal temperature between 50-80° C. A solution of water (148 kg) and chloroacetic acid (112 kg) is dosed to vessel 4 at an internal temperature between 50-80° C. and the chloroacetic acid vessel is flushed with water (108 kg) into vessel 4. The reaction mixture is heated to higher than 100° C. and is stirred at this temperature for more than 10 hours. The reactor content is then cooled to 75-85° C. The internal temperature of the reaction mixture is brought to 70-80° C., before 36% hydrochloric-acid (209 kg) is charged keeping the internal temperature between 70-80° C. The reaction mixture is cooled to 55-65° C. and stirred for at least 15 min before it is cooled to preferably 20° C. The suspension is isolated via centrifuge and the wet cake is flushed with water (756 kg). The product is dried under vacuum at maximum temperature of 60° C. to give 123.9 kg (63.1%) of crude dried product 10.

A vessel is charged with crude Methylsulfon 10 (109.4 kg) and acetonitrile (259.3 kg). The suspension is heated to reflux and stirring is continued for at least 30 min at reflux. The vessel content is cooled down from reflux temperature to an internal temperature between 13-23° C. The suspension is stirred at this temperature for at least 30 min. The product is isolated by centrifugation, the wet cake is washed with acetonitrile (109 kg). The product was dried under vacuum at a maximum temperature of 60° C. to afford 94.7 kg (86.5%)of dried product 10.

### Example 1 (Step 1): Preparation of phenylether (R)-3-OH (R)-5-(methylsulfonyl)-2-((1,1,1-trifluoropropan-2-l)oxy)benzoic acid

### Procedure A

n-BuLi in hexanes (10.6 L, 2.5 M solution, 26.4 mol) was slowly added to a solution of crude 9 (11.3 kg, 19.0% w/w, 13.7 mol) in THF keeping the internal temperature below 30° C. and the resulting mixture was then stirred at ambient temperature for 1 h. A solution of 10 (2.38 kg, 96.8% by weight; 10.6 mol), in THF (9.2 L) and NMP (2.3 L) was charged to the reactor originally containing 9 and n-BuLi solution, and the resulting mixture was stirred at reflux (63-65° C.) for a minimum of 1 h. The mixture was quenched with 2 N HCl (14.0 L, 28.0 mol) and concentrated by distillation at 40-45° C. under reduced pressure to a total volume of approximately 18 to 19 L. The mixture was next extracted with ethyl acetate (23.0 L), washed with 5% NaCl aqueous solution (7.0 kg) and distilled to a minimum stirrable volume (7 to 8 L). Methanol (18.0 L) was charged and the distillation continued until the volume was approximately 10 to 11 L. Water (9.0 L) was charged at 40-45° C. and the distillation continued until the volume reached approximately 6 to 7 L. The mixture was allowed to reach ambient temperature and the resulting solid was collected by filtration, rinsed sequentially with water (18.0 L) and heptane (18.0 L) and then dried in a vacuum oven at 45-50° C. for a minimum of 10 hours to give 2.9 kg (88.0% yield) of (R)-3-OH as a white solid: ¹H NMR (400 MHz, DMSO-d₆) δ 1.47 (d, J=6.4 Hz, 3H), 3.25 (s, 3H), 5.50 (m, 1H), 7.59 (d, J=8.8 Hz, 1H), 8.07 (dd, J=2.4, 8.8 Hz, 1H), 8.18 (d, J=2.4 Hz, 1H), 13.3 (br, 1H); ¹³C NMR (75 MHz, DMSO-d₆) δ 13.7, 44.1, 72.6 (q, J=23.8 Hz), 116.2, 124.0, 124.9 (q, J=209.5 Hz), 129.11, 130.5, 132.3, 134.4, 158.9, 166.2.

### Procedure B

The hydrogenation vessel 1 is charged with 3,3,3-trifluoropropenyl-2-acetate 7 (77 kg). A separate mobile stirring tank is charged with catalyst Rh(nbd)₂BF₄ (280 g), ligand t-Bu-SMS-Phos.2HBF₄ 8 (644 g), inertised with argon, and flushed with tetrahydrofuran (231 kg) into the hydrogenation vessel. Potassium-tert-butylate (20%) in THF (924 g) is charged into the separate mobile stirring tank, inertised with argon and flushed with tetrahydrofuran (42,5 kg) into the hydrogenation vessel. Hydrogenation is carried out at an internal temperature of 20 to 40° C. and p=1,5 bar hydrogen until hydrogenation is completed.

The Acetate 9 solution is filtered into a mobile tank using 17,2 kg THF to flush the hydrogenation vessel. Dimethylsulfoxide (149 kg) is charged into vessel 2 and heated to an inner temperature of 45-55° C. Then methylsulfon 10 (90 kg) is charged into vessel 2 at an internal temperature of 45-55° C. and stirred until a clear solution is obtained.

Then dimethylsulfoxide (98 kg), potassium hydroxide (84 kg) and tetrahydrofuran (121 kg) are charged into vessel 3 and the internal temperature is adjusted to 25-35° C. Then Acetate 9 solution (345 kg) is dosed into vessel 3 at an internal temperature of 25-35° C. and the transport container is flushed with tetrahydrofuran (18 kg) into vessel 3.

Then content of vessel 2 is dosed into vessel 3 at an internal temperature of 20 to 60° C. in 15-35 min. Vessel 2 is flushed with dimethylsulfoxide (50 kg) into vessel 3. The resulting solution is stirred for at least 60 min at an internal temperature of 45-60° C. Then the reaction solution is cooled to an internal temperature of 15-25° C. The reaction solution is filtered into vessel 4 via a filter cascade. The filters are flushed with tetrahydrofuran (81 kg) into vessel 4. The reaction solution in vessel 4 is heated to an internal temperature of 50-65° C. and solvents are distilled off at 50-65° C. inner temperature under vacuum (not lower than 100 mbar).

Water (180 kg) is added to vessel 4 at an internal temperature of 50-65° C., followed by addition of 36% HCl aq. (73 kg) keeping the internal temperature in a range of 50-65° C. and finally water (34 kg) is added keeping the internal temperature in the same range. The vessel content is stirred for 15-30min at 50-65° C. and water (146 kg) is dosed within at least 15 min at an internal temperature 50-65° C. The resulting mixture is stirred for 15-30 min and then cooled in a linear ramp to 15-25° C. in 60-90 min. The suspension is isolated via centrifuge and the wet cake is flushed with water (900 kg). The product is dried under vacuum at a maximum temperature of 60° C. to give 106 kg (82%). The EE-value of product (R)-3-OH is 99.38%.

### Example 2 (Step 2): Preparation of sodium salt (R)-3-ONa Sodium (R)-5-(methylsulfonyl)-2-((1,1,1-trifluoropropan-2-yl)oxy)benzoate

### Procedure A

Carboxylic acid (R)-3-OH (20.1 kg, 63.1 mol) was charged to a jacketed reactor under nitrogen followed by 2-propanol (225 L) and the resulting mixture was agitated at 70-75° C. for a minimum of 0.5 h (homogenous solution). A 50% sodium hydroxide solution (5.35 kg, 66.9 mol) was charged and stirring continued at 70-75° C. for a minimum of 1 h. A suspension of (R)-3-ONa seed crystals (0.2 kg, 0.59 mol) and 2-propanol (10.0 L) was added and the batch was stirred at 65-70° C. for a minimum of 2 h. The batch was then cooled linearly to 20-25° C. over a period of approximately 4 h. The resulting solid was collected by filtration, rinsed with 2-isopropanol (50 L) and then dried in a vacuum oven at 45-50° C. for a minimum of 10 hours to afford 18.6 kg of product (98.8% by weight) in 86.3% yield: ¹H NMR (400 MHz, DMSO-d₆) δ 1.42 (d, J=6.4 Hz, 3H), 3.16 (s, 3H), 5.32 (m, 1H), 7.25 (d, J=8.8 Hz, 1H), 7.71 (dd, J=2.4, 8.8 Hz, 2H), 7.87 (d, J=2.4 Hz, 1H); ¹³ C NMR (75 MHz, DMSO-d₆) δ 14.2, 44.3, 73.3 (q, J=23.0 Hz), 118.4, 125.4 (q, J=210.3 Hz), 127.2, 128.8, 134.5, 136.0.

### Procedure B

Vessel 1 is charged with Phenylether (R)-3-OH (140 kg) and isopropanol (964 kg). The mixture is heated to an internal temperature of 65-75° C. and stirring is continued until a clear solution is obtained. 45% Sodium hydroxide solution (40 kg) is added at an internal temperature of 65-75° C., then seeding crystals (0,5 kg) are added and stirring is continued at this temperature for 10-60 min. The vessel content is cooled down to 15-25° C. within 45-120 min. The product is isolated by centrifugation, the wet cake is washed with isopropanol (310 kg). The product is dried under vacuum at a maximum temperature of 80° C. to give 138.5 kg (92%). The EE-value of product 3-ONa is enriched to 99.82% compared to 99.38% at step (R)-3-OH.

### Example 3 (Step 3): Preparation of enolate (R,R)-12

Carboxylic acid (R,R)-13 (175 kg) is dissolved in THF (467 kg) and cooled to −20° C. A solution of MeLi (8% in diethoxymethane, 487 kg) is added at a temperature of −25 to −10° C. and the reaction mixture is stirred at this temperature for 3 h. The reaction mixture is charged to a solution of ammonium chloride (26 kg) and water (499 kg) at 10 to 35° C. THF is removed by vacuum distillation at a maximum temperature of 60° C. Methyl-tert-butyl ether (648 kg) is added to the residue and the phases are separated. The organic layer is concentrated by vacuum distillation at a maximum temperature of 60° C. THF (467 kg) is added to the residue and the solution is concentrated by vacuum distillation at a maximum temperature of 60° C. Lithium tert-butoxide (20% in THF, 309 kg) is added at −5 to 10° C. followed by an addition of ethyl trifluoroacetate (115 kg) at −5 to 10° C. The mixture is stirred for at least 60 min at 15 to 25° C. The reaction mixture is charged to a solution of ammonium chloride (18 kg) in water (333 kg) at 15 to 35° C. The organic solvents are removed by vacuum distillation at a maximum temperature of 60° C. Methyl-tert-butyl ether (882 kg) is added to the residue and the phases are separated. The organic layer is concentrated by vacuum distillation at a maximum temperature of 60° C. Methanol (455 kg) is added to the residue and seed crystals are added at a temperature of 30° C. The suspension is cooled to 20° C. and stirred at this temperature for 60 min. Water (980 kg) is added and stirred at 20° C. for 30 min. The suspension is cooled to 10° C. and stirred at this temperature for 60 min. The product is isolated by centrifugation, washed with water (320 kg) and then dried under vacuum at a maximum temperature of 60° C. to give 173.9 kg (69%).

### Example 4 (Step 4): Preparation of dihydroisoxazole (R,R)-14

Vessel 1 is charged with Enolate 12 (149 kg) and isopropanol (317 kg). The resulting suspension is stirred at 20-30° C. for around 20 min. An aqueous solution of hydroxylamine hydrochloride (180 kg, 23.2% aqueous solution) is dosed to the reaction mixture in 20-40 min maintaining the internal temperature between 20-30° C. The reaction mixture is stirred for preferable 3 h at 20-30° C. Then water (932 kg) is charged to vessel 1 over 20-35 min. The suspension is stirred for 12-15 h at 15-25° C. The suspension is isolated via centrifuge and the wet cake is flushed with water (394 kg). The product is dried under vacuum at a maximum temperature of 60° C. to give 129.1 kg (84.4%) of dried product.

### Alternative Synthesis Route

### Preparation of tert-Butyl (1R,5R)-1-acetyl-3-azabicyclo[3.1.0]hexane-3-carboxylate (enolate (R,R)-12)

Carboxylic acid (R,R)-13 (22.4 kg, 94.8%, 93.4 mol) and THF (105 L) were charged into a reactor under nitrogen. The mixture was cooled to −15±5° C. and a solution of MeLi in diethoxymethane (3.0 M, 71.6 L, 215 mol) was slowly charged keeping the internal temperature at −10 to −15° C. The batch was then stirred for an additional 2 h at −10° C. and upon completion a 5% solution of ammonium chloride (3.3 kg of ammonium chloride in 62 L of water) was charged. The mixture was distilled to the minimum stirrable volume at approximately 40-50° C. under reduced pressure to remove most of THF and DEM (diethoxymethane). t-Butyl methyl ether (149 L) was charged next and the mixture was stirred for 5-10 min at 20-25° C. The layers were allowed to settle, the bottom aqueous layer was discarded and the organic portion was washed with a 5% ammonium chloride solution (3.3 kg of ammonium chloride in 62 L of water). The mixture was then concentrated by distillation under reduced pressure at 40-45° C. to a minimum stirrable volume. Tetrahydrofuran (105 L) was charged. The mixture was then concentrated by distillation under reduced pressure at 40-45° C. to a minimum stirrable volume to give 30 kg of crude ketone (52.9% by weight solution, 75.2% yield) that was used in the subsequent chemical step. An analytical sample was prepared for characterization purposes by purification with silica gel chromatography: ¹H NMR (400 MHz, CDCl₃) δ 0.93-0.97 (m, 1H), 1.45 (s, 9H), 1.61 (dd, J=4.8, 8.4 Hz, 1H), 2.04-2.11 (m, 4H), 3.40-3.43 (m, 1H), 3.56-3.81 (m, 3H); ¹³C NMR (75 MHz, CDCl₃, mixture of rotamers) δ 19.4, 19.9, 25.6, 26.7, 27.4, 28.4, 28.6, 47.3, 47.4, 47.5, 47.6, 79.8, 154.9, 205.1. HRMS (ESI) calculated for [C₁₂H₂₀NO₃]⁺ 226.14377, observed 226.14385.

tert-Butyl (1R,5R)-1-(5-hydroxy-5-(trifluoromethyl)-4,5-dihydroisoxazol-3-yl)-3-azabicyclo [3.1.0]hexane-3-carboxylate ((R,R)-14): LiN(TMS)₂ (79.7 L of a 1.0 M solution in THF, 79.7 mol) was charged to a reactor under nitrogen and then cooled to −20 to −15° C. Crude ketone in THF (28.6 kg of a 52.4% solution in THF, 66.5 mol) was then slowly added keeping the internal temperature at or below −10° C. The mixture was stirred at −15 to −10° C. for approximately 1 hour, ethyl trifluoroacetate was added (11.9 L, 99.8 mol) slowly keeping the internal temperature at −15 to −5° C. and then the reaction mixture was allowed to reach ambient temperature (20-25° C.) over 2 h. The batch was cooled to 0-5° C. and 5% aqueous NH₄Cl (45.8 kg) was charged slowly keeping the internal temperature at or below 35° C. The mixture was concentrated by distillation under reduced pressure at 30-35° C. until a volume of approximately 83-85 L was reached. The mixture was then cooled to 20-25° C., MTBE (150 L) was charged and the batch was agitated at 20-25° C. for approximately 15 min. The layers were then allowed to separate and the aqueous layer was discarded. The organic layer was then washed with 5% aqueous NH₄Cl (45.8 kg) and then concentrated by distillation under reduced pressure at 35-40° C. until the volume was reduced to approximately 37-38 L. Methanol (150 L) was charged, the batch was filtered through a charcoal cartridge (12″×9″ activated charcoal) and after rinsing the filtration cartridge with methanol (60 L), the batch was concentrated by distillation under reduced pressure at 35-40° C. to a volume of approximately 80-85 L. The resulting solution containing crude diketone (protonated enolate (R,R)-12, (HRMS (ESI) calculated for [C₁₄H₁₈F₃NO₄]⁺ 322.12607, observed 322.12626) was then cooled to 20-25° C., a 47% solution of hydroxylamine hydrochloride (5.54 kg in 7.45 L of water) was charged and the mixture was stirred for 1 h. Water (90 L) was charged slowly over approximately 30 min, the mixture was stirred for 4 h and the resulting solid was collected by filtration. The filter cake was rinsed sequentially with water (60 L) and heptane (30 L) and then dried in a vacuum oven 45-50° C. for a minimum of 8 hours to give 22.4 kg (99.2% by weight, 99.3% yield) of (R,R)-14 product as a white solid: ¹H NMR (400 MHz, DMSO-d₆) δ 0.72 (m, 1H), 1.8 (obs m, 1H), 1.33 (s, 9H), 1.95-2.041 (m, 1H), 2.90 (m, 1H), 3.28-3.60 (m, 5H); ¹³C NMR (75 MHz, DMSO-d₆, mixture of rotamers) δ 16.0, 16.5, 23.6, 24.0, 24.4, 24.9, 25.9, 26.8, 42.8, 42.9, 47.7, 48.0, 48.4, 48.5, 48.6, 48.7, 79.3, 103.0, 103.1, 103.3, 103.4, 103.6, 118.7, 121.5, 124.3, 127.1, 154.4, 160.2, 160.3. HRMS (ESI) calculated for [C₁₄H₂₀F₃N₂O₄]⁺ 337.13697, observed 337.13696.

### Example 5 (Step 5): Preparation of isoxazole (R,R)-2 3-(1R,5R)-3-Azabicyclo[3.1.0]hexan-1-yl)-5-(trifluoromethyl)isoxazole hydrochloride ((R,R)-2)

Dihydroisoxazole (R,R)-14 (18.7 kg, 99.2% by weight, 55.2 mol) followed by acetonitrile (37.0 L) were charged to a reactor at ambient temperature. The mixture was heated to 35-40° C. and thionyl chloride was charged. The mixture was heated to 40-45° C. and stirred for 2 h. Isopropyl acetate (223 L) was charged and the mixture was concentrated by distillation under vacuum at 40-45° C. to the minimum stirrable volume to remove acetonitrile. The mixture was cooled to 20-25° C. and an additional 130 L of isopropyl acetate were charged followed by 60.3 kg of a 2 N sodium hydroxide solution. After stirring for 5-10 min the layers were allowed to settle, the aqueous layer was collected and discarded, and the organic layer was washed with water (18.5 L). The organic layer was filtered through a charcoal cartridge (12″×9″ activated charcoal) and after rinsing the filtration cartridge with isopropyl acetate (37.1 L), the batch was concentrated by distillation under reduced pressure at 35-45° C. to a volume of approximately 55 L.

Hydrochloric acid (5-6 N solution in isopropanol, 13.8 L, 82.7 mol) was then charged over 15 min keeping the internal temperature at approximately 35-40° C. The internal temperature was increased to 50-55° C. and seed crystals of isoxazole 2 (185 g) were added. The mixture was heated to 50-55° C. and heptane (148 L) was charged slowly over a period of approximately 2 hours. The mixture was cooled linearly to 20-25° C. and the resulting solid was collected by filtration to give 13.2 kg (94% yield) of product 2 as a white solid: ¹H NMR (400 MHz, DMSO-d₆) δ 1.48 (t, J=7.6 Hz, 1H), 1.73 (t, J=5.8 Hz, 1H), 2.33 (m, 1H), 3.40 (d, J=11.2 Hz, 1H), 3.47 (dd, J=4.0, 11.6 Hz, 1H), 3.64 (d, J=11.2, 1H), 3.70 (d, J=11.6 Hz, 1H), 7.52 (d, J=0.8 Hz, 1H), 10.0 (br, 2H); ¹³C NMR (75 MHz, DMSO-d₆) δ 15.6, 24.5, 25.8, 46.7, 46.9, 105.8, 118.2 (q, J=201 Hz), 157.2 (m), 164.9; HRMS (ESI) calculated for [C₉H₁₀F₃N₂O]⁺ 219.07397, observed 219.07415.

### Example 5b (Step 5): Preparation of isoxazole (R,R)-2 as Free Base 3-((1R,5R)-3-Azabicyclo[3.1.0]hexan-1-yl)-5-(trifluoromethyl)isoxazole ((R,R)-2 as Free Base)

Thionyl chloride (63 kg) was added in three portions to a stirred suspension of dihydroisoxazole (R,R)-14 (155 kg) in acetonitrile (367 kg) at 10-20° C. After each addition the reaction mixture was stirred for 1-2 hours at 10-20° C. Afterwards the reaction mixture was stirred for 5 h at 15-25° C. Then it was charged to water (450 kg) at 15-25° C. Acetonitrile was removed by vacuum distillation at a maximum temperature of 70° C. Afterwards MTBE (217 kg) was added, the phases were separated and the organic layer was discarded. Residual MTBE was removed from the aqueous layer by vacuum distillation at a maximum temperature of 50° C. After addition of water (503 kg), 45% aqueous NaOH (97 kg) was added at a temperature of 10-25° C. and a suspension was obtained. The suspension was cooled to 15° C. and stirred at this temperature for 60 minutes. The product was isolated by centrifugation, washed with water (580 kg) and then dried under vacuum at a maximum temperature of 30° C. to give 87.5 kg (87%).

### Example 6 (Step 6): Preparation of Compound 1 ([5-(methylsulfonyl)-2-{[(2R)-1,1,1-trifluoropropan-2-yl]oxy}phenyl]{(1R,5R)-1-[5-(trifluoromethyl)-1,2-oxazol-3-yl]-3-azabicyclo[3.1.0]hex-3-yl}methanone)

### Procedure A

Isoxazole (R,R)-2 (14.4 kg, 343 mol) and DMF (22.0 L) were charged under nitrogen into a reactor at ambient temperature under nitrogen. The internal temperature was adjusted to 10-15° C., a 50% solution of T3P in ethyl acetate (30.9 L, 51.8 mol) was charged over 30 min keeping the internal temperature at 10 to 15° C. and the mixture was then stirred for approximately 20 min. A solution of (R)-3-OH (11.0 kg, 254.6 mol) in DMF (17.6 L) was prepared and charged slowly over approximately 30 min into the reactor keeping the internal temperature at 10 to 15° C. The container with the (R)-3-OH/DMF solution was rinsed into the reactor with DMF (4.4 L) and the mixture was stirred for approximately 3 h. The internal temperature was adjusted to 10-15° C. and triethylamine (15.1 L, 108 mol) was slowly charged into the reactor over approximately 45 min keeping the internal temperature at 10-15° C. The mixture was stirred for 20 min, the temperature was adjusted to 20-25° C. and the mixture was stirred at 20-25° C. for 12 h. Water (55.0 L) was charged, the mixture was extracted with ethyl acetate twice (55.0 L per extraction) and the combined organic layer was washed first with 8.0% aq. NaHCO₃ (55.0 L) and then with water (55.0 L). The mixture was concentrated to approximately 30 L by distillation (40-50° C., reduced pressure), isopropanol (101 L) was added and the mixture was then concentrated to the minimum stirrable volume by distillation (50-55° C., reduced pressure). Additional isopropanol (101 L) was added and the mixture was then concentrated (50-55° C., reduced pressure) to give 73.4 kg of a solution containing 27.8% of product 1 (20.4 kg) by weight. Additional isopropanol (9.9 L) was charged to bring the concentration of Compound 1 and isopropanol solution to 25% by weight and the temperature was increased to 65-75° C. Heptane (51.0 L) was charged followed by a suspension of 1 seed crystals (154 g) in heptane (1.54 L) keeping the temperature at 65-75° C. The mixture was then cooled to ambient temperature (20-25° C.) linearly over a period of 6 h, the resulting solid was collected by filtration and the reactor and cake were washed with heptane (44.0 L). The material was then dried under reduced pressure in a vacuum oven for 12 h at 45-50° C. to afford 19.1 kg of product (98.7% by weight, 85.2% yield) as a white solid: ¹H NMR (400 MHz, CDCl₃, 1:1 mixture of rotational isomers at ambient temperature) δ 1.15 (t, J=5.2 Hz, 1H), 1.42 (q, J=7.6 Hz, 1H), 1.56 (m, 3H), 1.99-2.12 (m, 2H), 3.07 (d, J=1.6 Hz, 3H), 3.34 (d, J=10.8 Hz, 0.5H), 3.55 (d, J=11.2 Hz, 0.5 H), 3.60-3.68 (m, 1H), 3.91-4.04 (m, 1H), 4.25 (d, J=12.0 Hz, 0.5H), 4.47 (d, J=12.0 Hz, 1H), 4.86 (m, 1H), 6.50 (d, J=0.8 Hz, 0.5H) 6.38 (d, J=0.8 Hz, 0.5H), 7.13 (d, J=8.8 Hz, 1H), 7.90 (t, J=2.4 Hz, 1H), 7.98 (dt, J=8.8 Hz, 2.4 Hz, 1H); ¹³C NMR (75 MHz, CDCl₃, 1:1 mixture of rotational isomers at ambient temperature) δ 13.7, 17.4, 23.2, 24.2, 25.1, 25.8, 44.6, 47.3, 48.6, 49.6, 72.7, 73.0, 73.3, 73.6, 102.7, 103.2, 114.1, 116.3, 119.0, 122.5, 125.3, 18.3, 129.1, 130.6, 135.0, 155.9, 158.9, 159.3, 163.8, 165.7; HRMS (ESI) calculated for [C₂₀H₁₉F₆N₂O₅S]⁺ 513.09134, observed 513.09164.

### Procedure B

Thionyl chloride (17.40 kg, 146.3 mol) was slowly added to sodium salt (R)-3-ONa (19.55 kg, 58.49 mol) in toluene (61.0 kg) at 55° C. The mixture was heated to 80° C. within 30 min and stirred for at least 30 min before being cooled to 50° C. Toluene was removed by distillation at 50° C. to a minimum stirrable volume. Toluene (101.7 kg) was added and distilled off again. Me-THF (28.54 kg) was added.

To a solution of isoxazole (R,R)-2 (12.75 kg, 58.44 mol, as free base) in Me-THE (65.57 kg) in a separate container triethylamine (7.12 kg, 70.36 mol) was added. The resulting solution was slowly added to the acid chloride solution to keep temperature below 30° C. The solution was further stirred for at least 30 min. Water (117.3 kg) was added at 20° C. and further stirred for at least 15 min. The organic layer was concentrated to a minimum stirrable volume by distillation at 50° C. Isopropanol (60.6 kg) was added and distilled off (two times). The mixture was heated to 70° C., stirred for 15 min and slowly charged with isopropanol/n-heptane (1/5 v/v, 206.6 kg). Seed crystals (0.12 kg) were added and the mixture was cool to 55° within 1.5 h followed by cooling to 20° C. The resulting solid was collected, washed with isopropanol/heptane and dried under reduced pressure to provide Compound 1 (23.95 kg of crude product as a white solid, 80% mass recovery).

### Procedure C

Thionyl chloride (43.7 kg) is added to sodium salt (R)-3-ONa (80.5 kg) in toluene (453 kg) at 55-80° C. in 10-30 minutes. The mixture is heated to 80° C. within 30 min and stirred for 3 hours at this temperature. The reaction mixture is concentrated by vacuum distillation at max. 70° C. Toluene (418 kg) is added and distilled off again. Then 2-MeTHF (193 kg) is added. In a separate reactor 29.5 kg triethylamine are added to a solution of isoxazole 2 (50.0 kg) in 2 MeTHF (260 kg) at 15-25° C. The resulting solution is slowly added to the acid chloride solution at 15-35° C. and the reaction mixture is stirred at 20° C. for 40 minutes. Then it is added to water (724 kg). Then the phases are separated and the organic layer is concentrated under vacuum at max. 70° C. Afterwards 2-propanol (652 kg) is added and the reaction mixture is again concentrated under vacuum at max. 70° C., followed by addition of another portion of 2-propanol (652 kg). n-Heptane (695 kg) is added to the solution at 60-70° C. followed by seed crystals (0.5 kg). The resulting suspension is cooled to 20° C. in 2 hours. Then it is warmed to 65° C. and stirred at this temperature for at least 30 minutes, before it is again cooled to 20° C. in 2 hours. The product is isolated by centrifugation, washed with n-heptane (360 kg) and dried at max. 60° C. to give 99.8 kg (85%) of Compound 1.

1. A process for preparing Compound 1, comprising
reacting phenylether (R)-3-OH, its sodium salt (R)-3-ONa, or the corresponding acid chloride ((R)-3-Cl) with isoxazole 2, or the corresponding HCl adduct (R,R)-2.HCl, to provide Compound 1 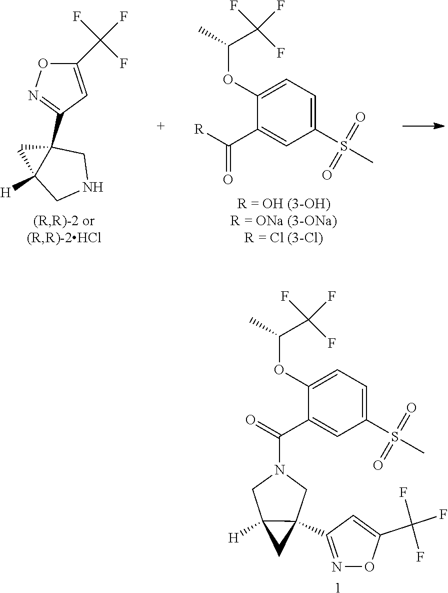
wherein R is OH, ONa, or Cl.

2. The process according to claim 1, further comprising:
adding propanephosphonic acid anhydride in ethyl acetate to a solution of phenylether (R)-3-OH or (R)-3-ONa followed by addition of a solution of isoxazole 2.

3. The process according to claim 1, wherein the acid chloride (R)-3-Cl is reacted with isoxazole 2 in solution.

4. The process according to claim 3, further comprising:
reacting the sodium salt of phenylether (R)-3-ONa with thionyl chloride or oxalyl chloride to provide the acid chloride (R)-3-Cl.

5. The process according to claim 4, wherein a solution of isoxazole 2 in Me—THE is added to a solution of the acid chloride (R)-3-Cl.

6. A process for preparing phenylether (R)-3-OH 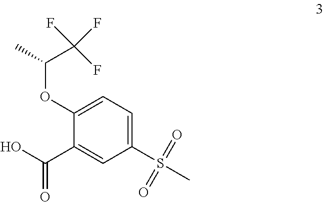 [SMILES: C[C@@H](Oc1ccc(S(C)(=O)=O)cc1C(=O)O)C(F)(F)F]
comprising:
reacting acetate 9 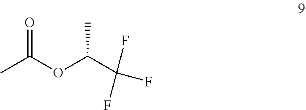
with methyl sulfone 10 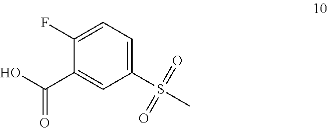
in the presence of potassium hydroxide to provide (R)-3-OH.

7. The process according to claim 6, wherein the reaction is performed using a solvent selected from the group consisting of dimethyl sulfoxide or a mixture of dimethyl sulfoxide and tetrahydrofuran.

8. The process according to claim 6, further comprising:
reacting fluorobenzoic acid with chlorosulfonic acid followed by sodium sulfite and chloroacetic acid to provide methylsulfone 10.

9. The process according to claim 6, further comprising:
reacting phenylether (R)-3-OH with sodium hydroxide in isopropanol to provide the sodium salt (R)-3-ONa.

10. The process according to claim 6, further comprising:
converting 3,3,3-trifluoropropenyl-2-acetate (7) in a Rh catalyzed hydrogenation reaction to acetate 9 and subjecting the crude hydrogenation solution to the process according to claim 6.

11. A process for preparing enolate (R,R)-12 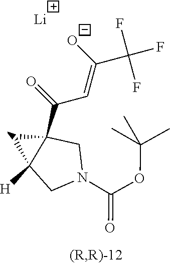
comprising:
reacting carboxylic acid (R,R)-13 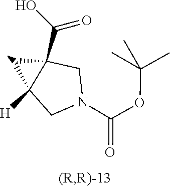
with methyl lithium and ethyl trifluoroacetate to provide (R,R)-12.

12. The process according to claim 11, further comprising quenching the product of the reaction of (R,R)-13 with methyl lithium with aq. ammonium chloride followed by deprotonation with lithium tert-butoxide or lithium bis (trimethylsilyl)amide before reacting with ethyl trifluoroacetate.

13. The process according to claim 11, further comprising:
reacting (R,R)-12 with hydroxylamine to provide dihydroisoxazole (R,R)-14

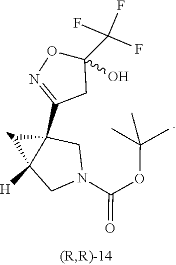

14. A process for preparing isoxazole (R,R)-2, 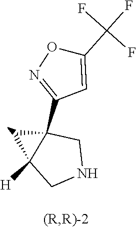
comprising:
reacting dihydroisoxazole (R,R)-14 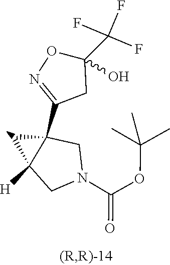
with a reagent selected from the group consisting of thionyl chloride and oxalyl chloride to provide (R,R)-2.

15. The process according to claim 1, further comprising:
reacting dihydroisoxazole (R,R)-14 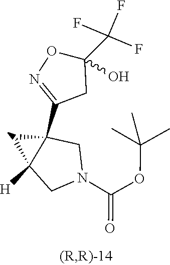
with a reagent selected from the group consisting of thionyl chloride and oxalyl chloride to provide (R,R)-2.

16. The process according to claim 1, further comprising:
reacting acetate 9 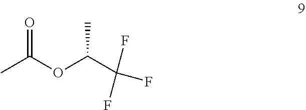
with methyl sulfone 10 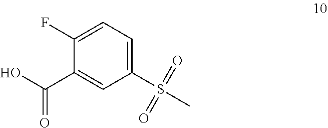
in the presence of potassium hydroxide to provide (R)-3-OH.

17. A compound selected from the group consisting of: 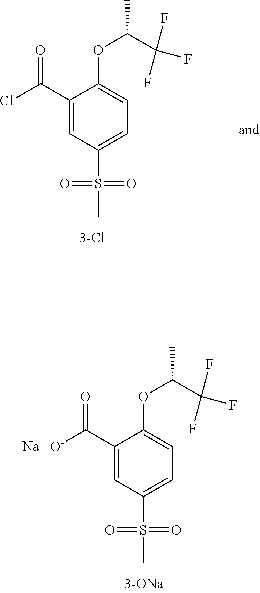 [SMILES: C[C@@H](Oc1ccc(S(C)(=O)=O)cc1C(=O)Cl)C(F)(F)F ; [Na+] ; C[C@@H](Oc1ccc(S(C)(=O)=O)cc1C(=O)[O-])C(F)(F)F]
